SEQUENCE LISTING (4 sequences)

SEQ ID NO: 1 - DNA, 26 bp, Artificial Sequence
  Artificial primer
gaaccactgctactcggcatctggtc

SEQ ID NO: 2 - DNA, 23 bp, Artificial Sequence
  Artificial primer
cagaaatgtggctatccttcacc

SEQ ID NO: 3 - DNA, 27 bp, Artificial Sequence
  Artificial primer
gacgatcctgctctgattacccatcgc

SEQ ID NO: 4 - DNA, 22 bp, Artificial Sequence
  Artificial primer
tcagatcctgccacctgtccta

(19) United States
(12) Patent Application Publication
(10) Pub. No.: US 2011/0217301 A1
(43) Pub. Date: Sep. 8, 2011

(54) METHODS FOR TREATING OR SCREENING FOR COMPOUNDS FOR THE TREATMENT OF SEPSIS

(75) Inventor: ROBERT J. FREISHTAT, POTOMAC, MD (US)

(73) Assignee: THE CHILDREN'S RESEARCH INSTUTE, WASHINGTON, DC (US)

(21) Appl. No.: 13/081,166

(22) Filed: Apr. 6, 2011

Related U.S. Application Data
(63) Continuation-in-part of application No. 12/644,901, filed on Dec. 22, 2009.
(60) Provisional application No. 61/321,397, filed on Apr. 6, 2010.

(51) Int. Cl. A61K 39/395; A61K 38/12; A61K 31/4465; A61K 31/4365; A61K 31/343; A61K 31/19; A61K 31/192; A61K 31/60; A61K 31/235; A61K 31/4035; A61K 31/519; A61K 31/444; A61K 31/18; A61P 7/02; A61P 29/00; A61P 9/10; C12N 5/071; C12Q 1/37

(52) U.S. Cl. 424/133.1; 514/1.4; 514/331; 514/301; 514/468; 514/557; 514/569; 514/165; 514/548; 514/162; 514/416; 514/262.1; 514/332; 514/604; 435/375; 435/23

(57) ABSTRACT

The present invention relates to a method for treating and screening for compounds for the treatment of sepsis. More specifically, the treatment and screening methods are based on the discovery that granzyme B containing platelets (GzmB-platelet) causes apoptosis by direct contact with cells.

This application is a continuation-in-part of U.S. patent application Ser. No. 12/644,901, filed Dec. 22, 2008, which is incorporated herein by reference; and claims the priority of U.S. Provisional Patent Application No. 61/321,397, filed Apr. 6, 2010, which is also incorporated herein by reference.

### FIELD OF THE INVENTION

The present invention relates to a method for treating and screening for compounds for the treatment of sepsis. More specifically, the treatment and screening methods are based on the discovery that contact between granzyme B containing platelets (GzmB-platelet) causes apoptosis in a contact dependent manner.

### BACKGROUND OF THE INVENTION

Despite several decades worth of advances in antimicrobials, critical care, and organ support modalities (Hotchkiss et al., N Engl J Med 2003; 348:138-150; Russell, N Engl J Med 2006; 355:1699-1713), mortality rates from sepsis have remained largely unchanged at about 40% (Angus et al., Crit. Care Med 2001; 29:1303-1310). In fact, sepsis is responsible for 215,000 deaths annually in the US, which is akin to mortality from acute myocardial infarction (Angus et al.), making it the 10^{th} leading cause of death (Kochanek et al., Natl Vital Stat Rep 2004; 52:1-47). A recent paradigm shift indicates sepsis-related mortality results in part from immunodeficiency secondary to profound lymphoid apoptosis (Hotchkiss et al., Nat Rev Immunol 2006; 6:813-822). Indeed, this apoptosis is considered a diagnostic hallmark of progressive sepsis and multiple organ dysfunction. However, the etiology of the apoptosis is unknown.

Sepsis is characterized by a whole-body inflammatory state caused by infection. In systemic inflammations, as in the case of sepsis, the inflammation-specific reaction cascades spread in an uncontrolled manner over the whole body and become life-threatening in the context of an excessive immune response. A modern definition for sepsis is given in Levy et al. (Critical Care Medicine 31(4):1250-1256, 2003).

The inflammatory processes are controlled by a large number of substances, which are predominantly of a protein or peptide nature, or are accompanied by the occurrence of certain biomolecules. The endogenous substances involved in inflammatory reactions include, particularly, cytokines, mediators, vasoactive substances, acute phase proteins and/or hormonal regulators. The inflammatory reaction is a complex physiological reaction in which both endogenous substances activating the inflammatory process (e.g. TNF-α) and deactivating substances (e.g. interleukin-10) are involved. Current knowledge about the occurrence and the possible role of individual groups of endogenous inflammation-specific substances is disclosed, for example, in Beishuizen et al. (Advances in Clinical Chemistry 33:55-131, 1999); and Gabay et al. (The New England Journal of Medicine 340(6):448-454, 1999, 448-454).

For diagnostic purposes, the reliable correlation of disease with the respective biomarker is of primary importance, without there being any need to know its role in the complex cascade of the endogenous substances involved in the inflammatory process. U.S. Pat. No. 5,639,617 to Bohuon discloses the peptide procalcitonin as a marker of sepsis. U.S. Pat. No. 6,756,483 to Bergmann et al. discloses a shortened procalcitonin, containing amino acids 3-116 of the complete procalcitonin peptide, as the form that is actively involved in inflammatory processes and thus sepsis.

Other markers for sepsis include carbamoyl phosphate synthetase 1 (CPS1) or its N-terminal fragments (U.S. Pat. No. 7,413,850); CD25, CD11c, CD33, and CD66b leucocytes (U.S. Pat. No. 5,830,679); 3-chlorotyrosine or 3-bromotyrosine (U.S. Pat. No. 6,939,716); and CSaR (U.S. Pat. No. 7,455,837).

Many patients with septicemia or suspected septicemia exhibit a rapid decline over a 24-48 hour period. Thus, rapid methods of diagnosis and treatment delivery are essential for effective patient care. Clearly, there remains a need for agents capable of diagnosing and treating sepsis.

### SUMMARY OF THE INVENTION

Studies of sepsis have demonstrated accumulation of platelets in spleen and other end organs (Shibazaki et al., Infect Immun 1996; 64:5290-5294; Drake et al., Am J Pathol 1993; 142:1458-1470). Further, activated platelet-derived microparticles have cytotoxic activity toward vascular endothelium (Azevedo et al., Endocr Metab Immune Disord Drug Targets 2006; 6:159-164; Gambim et al., Crit. Care 2007; 11:R107; Janiszewski et al., Crit. Care Med 2004; 32:818-825) and smooth muscle (Janiszewski et al.). However, platelets are anucleate, having only cytoplasmic components imparted by megakaryocytes residing in the bone marrow, and are incapable of de novo gene transcription. Thus, these previous studies assumed that changes in platelet function were at the post-transcriptional level. Platelets do contain reservoirs of mRNA, and a number of studies have reported the transcriptome of human platelets using mRNA profiling (Raghavachari et al., Circulation 2007; 115:1551-1562; Dittrich et al., Thromb Haemost 2006; 95:643-651; Hillmann et al., J Thromb Haemost 2006; 4:349-356; Ouwehand et al., J. Thromb Haemost 2007; 5 Suppl 1:188-195). It has also been established that platelets regulate translation of their transcriptome in response to external stimuli (Weyrich et al., Blood 2007; 109:1975-1983; Weyrich et al., Proceedings of the National Academy of Sciences 1998; 95:5556-5561; Zimmerman et al., Arterioscler Thromb Vasc Biol 2008; 28:s17-24). However, no studies have shown acute changes in platelet mRNA pools as a function of a systemic stimulus, such as experimental or clinical sepsis.

Through genome-wide mRNA analysis, the present inventor has discovered that granzyme B is upregulated in platelets of subjects with sepsis and that the amount of granzyme B in the platelets directly corresponds to the severity of sepsis. Accordingly, this application relates to methods for the diagnosis, detection, or prognosis of sepsis, which are more sensitive and reliable than the tests of the prior art. The invention also relates to methods of treating or preventing apoptosis, and thus sepsis, by preventing ganzyme B-platelets from binding to cells.

The present invention provides methods for detecting or diagnosing or prognosticating sepsis. The methods comprise determining the presence or amount of granzyme B in platelets of an individual having or suspected of having sepsis. The presence of granzyme B (above a background level) indicates the presence of sepsis; and the amount of granzyme B directly correlates with the severity of the disease (the higher the concentration the more severe the disease).

The present invention further provides methods for monitoring the treatment of an individual with sepsis. The methods comprise administering a pharmaceutical composition to an individual suffering from sepsis, and determining the presence or amount of granzyme B in platelets of the individual. The treatment is considered successful if the amount of granzyme B decreases over the course of treatment. Treatment, however, should continue until the granzyme B amount decreases to background level or is non-detectable.

The present invention further provides methods for screening for an agent capable of modulating the onset or progression of sepsis. The methods comprise exposing an individual suffering from sepsis to the agent, and determining the presence or amount of granzyme B in platelets of the individual. The agent is considered capable of modulating the onset or progression of sepsis if, upon the administration of the agent, the amount of granzyme B decreases over the course of treatment or reduces to a background level.

In embodiments of the present invention, amount of granzyme B is determined by detecting granzyme B gene product in platelets using immunoassays, nucleic acid analysis, preferably mRNA, or substrate degradation. Gene products as recited herein can be nucleic acid (DNA or RNA) and/or proteins. In the case of DNA and RNA, detection can occur, for example, through hybridization with oligonucleotide probes. In the case of proteins, detection can occur, for example, though various protein interaction, such as specific binding reaction (e.g. immunoassay) and substrate degradation.

A sample for granzyme B determination can be obtained by withdrawing blood from the individual. In an embodiment, the platelets in the blood sample can be lysed and the granzyme B released from the platelets can be assayed. Alternatively, the platelets can be stained using, e.g. an immunostain targeting granzyme B, and stained cells can be observed using, e.g. hemocytometry techniques known in the art.

The serum test of the present invention can be used alone or in conjunction with the other diagnostic methods known in the art, such as the markers disclosed previously in the Background of the Invention.

The present invention also provides methods for preventing or reducing apoptosis of a cell by preventing or reducing contact of granzyme B-platelets (GzmB-platelets) with the cell. The methods involve contacting the cell with a compound that is effective to prevent or reduce the contact of the cell with GzmB-platelets.

The present invention further provides methods for treating or preventing sepsis by preventing or reducing contact of GzmB-platelets with cells of an organ. The methods involve administering to a septic animal a compound effective to prevent or reduce platelet aggregation, and thus, preventing interaction between GzmB-platelets and cells.

The compounds effective to prevent platelet aggregation include, but are not limited to, GP2a3b antagonists, ADP receptor/P2Y12 inhibitors, prostaglandin analogues (PGI2), COX inhibitors, thromboxane inhibitors, or phosphodiesterase inhibitors. Examples of GP2a3b antagonists are epifibitide, tirofiban, and abciximab. ADP receptor/P2Y12 inhibitors can be thienopyridines, such as clopidogrel, prasugrel, and ticlopidine. Examples of prostaglandin analogue include beraprost, prostacyclin, iloprost, and treprostinil. Examples of COX inhibitors are asprin, aloxiprin, carbasalate calcium, indobufen, and triflusal. Examples of thromboxane inhibitors include thromboxane synthase inhibitors such as dipyridamole and picotamide; and receptor antagonist such as terutroban. Other compounds effective to reduce apoptosis include anagrelide, heparin, cilostazol, and dipyridamole.

The present invention additionally relates to ex vivo methods for screening for drug candidates to treat sepsis. The methods involve contacting a candidate agent with a cell suspension or culture to from a mixture, and adding GzmB-platelets to the mixture. The suspension is then observed to determine whether apoptosis is reduced when compared to a control (mixture of the suspension or culture with GzmB-platelets without the candidate compound). If apoptosis is reduced, the agent is considered a drug candidate for the treatment of sepsis, and further study is recommended for the drug candidate. Alternatively, the methods involve contanting GzmB-platelets with the candidate agent. If the candidate agent is effective in preventing platelet aggregation when compared to a control (GzmB-platelets alone without the candidate agent), then the agent is considered a drug candidate for the treatment of sepsis.

### BRIEF DESCRIPTION OF THE DRAWINGS

FIG. 1 shows classification of sepsis severity via unsupervised clustering of comprehensive clinical and laboratory data. Data collected over 72 hours on children and adolescents (n=17) admitted to our tertiary care pediatric ICU with a presumed diagnosis of sepsis were input into Hierarchical Clustering Explorer (HCE). Variables input included the following at 0, 24, 48, and 72 hours: Temperature; heart rate; respiratory rate; systolic, diastolic, and mean arterial blood pressure; Glasgow coma score; blood pH, pCO₂, O₂, and base excess; white blood cell count; absolute neutrophil, lymphocyte, and monocytes counts; blood hemoglobin and platelet count; prothrombin and activated partial thromboplastin times; serum sodium, potassium, chloride, glucose, creatinine; and blood urea nitrogen. Similarities between these phenotypes are reflected in the cluster shown with shorter bars representing more similarity. These results were used to classify the septic participants as severe (n=6) and moderate (n=7) as shown by the overlaid boxes.

FIG. 2 shows platelet granzyme B mRNA expression reflects megakaryocyte expression in septic mice. Platelets do not have transcriptional machinery, therefore changes in platelet granzyme B mRNA expression in septic mice (n=12) were measured simultaneously in autologous megakaryocytes. Results of this qRT-PCR analysis show good correlation between increasing megakaryocyte and platelet granzyme B mRNA expression over time.

FIG. 3 shows flow cytometric measurement of intracellular granzyme B expression in platelets from septic and healthy children. Citrated whole blood was gated on CD61* platelets. Intracellular granzyme B was measured in healthy children (n=10) and septic children we classified as severe (n=1) and moderate (n=3) one and three days following admission. Shown are results from the child with severe disease showing platelet granzyme B expression at both day one (49.7%) and day three (44.3%) compared to the isotype control. Only one of the moderate septic subjects expressed any granzyme B and only at day three (24.0%). There was no measurable intracellular granzyme B in platelets from the control children.

FIG. 4 shows that platelets harvested from septic mice induce apoptosis in control CD4⁺ splenocytes except in the absence of granzyme B. Percent apoptosis was significantly higher in splenocytes co-incubated with platelets harvested from septic wild-type (i.e. C57BL6) mice than with platelets from healthy wild-type mice and splenocytes without platelets. Repeat experiments with platelets from septic granzyme B null (−/−) mice (i.e. B6.129S2-Gzmb^{tmlLey}) showed a complete lack of induced splenocyte apoptosis. Further platelet activation with recombinant TNFα under any of these conditions did not alter lymphotoxic capacity.

FIG. 5 shows sepsis survival and severity in wild type and GzmB null mice in a rapidly fatal CLP model. A. GzmB null (−/−) mice had lower sepsis scores than wild type mice at every time point. For example, at 22 hours, the mean±SEM wild-type score was 9±0.8 while the granzyme B null score was 6.8±0.7 (P=0.04) B. Kaplan-Meier survival curve for WT and GzmB null (−/−) mice in hours after CLP. GzmB null (−/−) mice survived longer following CLP than wild type mice (P=0.0019 by Cox Proportional Hazard Regression).

FIG. 6 shows platelet granzyme B apoptosis surveyed by TUNEL in spleen, lung, and kidney. Representative frozen sections of end organs [i.e. spleen (a), lung (b), and kidney (c)] from wild type (left) and granzyme B null mice (right) were stained for apoptosis with a TUNEL-based assay (TACS® 2 TdT In Situ Apoptosis Detection Kits, Trevigen, Gaithersburg, Md.). Increased brown staining, evident of apoptosis, is seen in the wild type spleens, lungs and kidneys. While the granzyme B null kidneys show apoptosis, there is no staining in the granzyme B null spleens and lungs. No apoptosis was noted in either set of heart and liver sections and is therefore not shown here. Photomicrographs were taken at 10× and 20× magnification.

FIG. 7 shows that platelet induced splenocyte apoptosis is contact dependent and perforin independent. A. Platelets harvested from septic mice induce apoptosis in control CD4+ splenocytes in the absence of perforin. Percent apoptosis was significantly higher in splenocytes co-incubated with platelets harvested from septic wild type (i.e. C57BL6) mice (n=5) than with platelets from healthy wild type mice (n=5) and splenocytes without platelets. Repeat experiments with platelets from septic perforin null mice (i.e. C57BL/6-PfptmlSdz) showed no reduction in induced splenocyte apoptosis. B. Direct platelet contact is necessary for GzmB mediated apoptosis. Incubation across a dividing semi-permeable (0.4 μm) membrane reduced splenocyte apoptosis (10.3±3 vs. 5.6±2.6; p<0.01) to a rate indistinguishable from non-platelet treated controls (5.6±2.5%; p=NS). Apoptotic splenocytes were almost entirely caspase+(i.e. >98%).

FIG. 8 shows that Eptifibatide but not anti-CD62P reduces septic platelet-induced splenocyte apoptosis ex vivo. A. Representative flow cytometry staining of CD4+ splenocytes for pan-caspase FLICA (Y axis) vs. TUNEL (X axis) in presence of (left-to-right) no platelets, septic platelets, septic platelets with anti-62P antibody, or septic platelets with eptifibatide. B. Shown is the mean±SEM percent of septic platelet-induced splenocyte and CD4+ splenocyte apoptosis (i.e. TUNEL+ and pan-Caspase+) compared between pre-treatment conditions (i.e. eptifibatide and anti-CD62P). These results were normalized to the level of apoptosis in splenocytes incubated with untreated septic platelets (dashed line). Both splenocytes overall and CD4+ splenocytes showed a significant reduction (p values <0.05) in apoptosis when platelets were pre-treated with eptifibatide. Pre-treatment with an anti-CD62P monoclonal antibody did not significantly alter platelet-induced splenocyte apoptosis.

### DETAILED DESCRIPTION OF THE PREFERRED EMBODIMENT

Many biological functions are accomplished by altering the expression of various genes through transcriptional (e.g., through control of initiation, provision of RNA precursors, RNA processing, etc.) and/or translational control. For example, fundamental biological processes such as cell cycle, cell differentiation and cell death, are often characterized by the variations in the expression levels of an individual gene or group of genes.

Changes in gene expression also are associated with pathogenesis. For example, the lack of sufficient expression of functional tumor suppressor genes and/or the over expression of oncogene/protooncogenes could lead to tumorgenesis or hyperplastic growth of cells (Marshall (1991) Cell 64:313-326; Weirlberg (1991), Science 254:1138-1146). Thus, changes in the expression levels of a particular gene or group of genes (e.g., oncogenes or tumor suppressors) serve as signposts for the presence and progression of various diseases.

Monitoring changes in gene expression may also provide certain advantages during drug screening development. Often drugs are screened and prescreened for the ability to interact with a major target without regard to other effects the drugs have on cells. Often such other effects cause toxicity in the whole animal, which prevents the development and use of the potential drug.

The present inventor has identified granzyme B in platelets as a marker associated with sepsis. Changes in granzyme B in platelets can also provide useful markers for diagnostic uses as well as markers that can be used to monitor disease states, disease progression, drug toxicity, drug efficacy and drug metabolism. Specifically, the present inventor has discovered a direct correlation between the upregulation of granzyme B in platelets and the presence of sepsis. The amount of granzyme B present also directly correlates with the severity of sepsis.

### Use of Granzyme B in Platelets as Diagnostics

As described herein, the granzyme B in platelets may be used as diagnostic markers for the detection, diagnosis, or prognosis of sepsis. For instance, a sample from a patient may be assayed by any of the methods described herein or by any other method known to those skilled in the art, and the expression levels of granzyme B in platelets may be compared to the expression levels found in normal platelets (platelets in individuals without sepsis) or to the background levels of granzyme B. The expression levels of granzyme B in platelets that substantially resemble an expression level from the serum of normal or of diseased individuals may be used, for instance, to aid in disease diagnosis and/or prognosis. Comparison of the granzyme B levels in platelets may be done by a researcher or a diagnostician or may be done with the aid of a computer and databases.

In general, the background amount of granzyme B in platelets is not detectable; thus, any detectable levels of granzyme B indicate the presence of sepsis. However, depending on the assay used, it is important to determine the background granzyme B levels to properly make a diagnosis. In general, severe sepsis is indicated if greater than about 40% of platelets express granzyme B; moderate sepsis exists if about 20-40% of platelets express granzyme B.

### Use of Granzyme B in Platelets for Drug Screening

According to the present invention, granzyme B levels in platelets may be used as markers to evaluate the effects of a candidate drug or agent on treating septic patients. A patient suffering from sepsis is treated with a drug candidate and the progression of the disease is monitored over time. This method comprises treating the patient with an agent, periodically obtaining samples from the patient, determining the levels or amounts of granzyme B in platelets from the samples, and comparing the granzyme B levels over time to determine the effect of the agent on the progression of sepsis.

Alternatively, the present invention also provides ex vivo methods for screening for drug candidates for the treatment of sepsis. The method comprises treating a cell culture with an agent, and adding GzmB-platelets to the treated culture. The cells in the culture are observed to determine whether apoptosis is reduced when compared to a control (cell culture mixed with GzmB-platelets without the agent). If apoptosis is reduced, the agent is considered a drug candidate for treating sepsis and should be further studied for safety and effectiveness. Alternatively, the methods involve contanting GzmB-platelets with the candidate agent. If the candidate agent is effective in preventing platelet aggregation when compared to a control (GzmB-platelets alone without the candidate agent), then the agent is considered a drug candidate for the treatment of sepsis. The ex vivo methods are especially useful in high throughput screening for identifying drug candidates, such as the system disclosed in U.S. Pat. No. 7,285,411, which is incorporated herein by reference.

The candidate drugs or agents of the present invention can be, but are not limited to, peptides, small molecules, vitamin derivatives, as well as carbohydrates. Dominant negative proteins, DNA encoding these proteins, antibodies to these proteins, peptide fragments of these proteins or mimics of these proteins may be introduced into the patient to affect function. “Mimic” as used herein refers to the modification of a region or several regions of a peptide molecule to provide a structure chemically different from the parent peptide but topographically and functionally similar to the parent peptide (see Grant (1995), in Molecular Biology and Biotechnology, Meyers (editor) VCH Publishers). A skilled artisan can readily recognize that there is no limit as to the structural nature of the candidate drugs or agents of the present invention.

### Use of Granzyme B in Platelets for Monitoring Disease Progression

As described above, the expression of granzyme B in platelets may also be used as markers for the monitoring of disease progression, for instance, the development of sepsis. For instance, a sample from a patient may be assayed by any of the methods described herein, and the expression levels of granzyme B in platelets may be compared to the expression levels found in uninfected individuals. The levels of granzyme B in platelets can be monitored over time to track progression of the disease. The present methods are especially useful in monitoring disease progression because the granzyme B expression in platelets is proportional to the severity of the disease. Comparison of the granzyme B expression levels may be done by researcher or diagnostician or may be done with the aid of a computer and databases.

### Assay Formats

The upregulation of granzyme B in platelets is manifest at both the level of messenger ribonucleic acid (mRNA) and protein. It has been found that increased granzyme B in platelets, determined by either mRNA levels, or biochemical measurement of protein levels, is associated with sepsis.

In an embodiment of the present invention, serum granzyme B levels are detected by immunoassays. Generally, immunoassays involve the binding of granzyme B and anti-granzyme B antibody. The presence and amount of binding indicate the presence and amount of granzyme B present in the sample. Examples of immunoassays include, but are not limited to, ELISAs, radioimmunoassays, immunoblots, and immuinostaining, which are well known in the art. The antibody can be polyclonal or monoclonal and is preferably labeled for easy detection. The labels can be, but are not limited to biotin, fluorescent molecules, radioactive molecules, chromogenic substrates, chemi-luminescence, and enzymes.

In an embodiment, ELISA, based on the capture of granzyme B by immobilized monoclonal anti-granzyme B antibody followed by detection with biotinylated polyclonal anti-granzyme B antibody, is used to detect serum granzyme B. In this system, the wells of a multi-well plate are coated with the monoclonal antibody and blocked with, e.g. milk or albumin. Samples are then added to the wells and incubated for capture of granzyme B by the monoclonal antibody. The plate may then be detected with the polyclonal antibody and strepavidine-alkaline phosphatase conjugate.

In another embodiment, granzyme B levels can be detected by measuring nucleic acid levels in the serum, preferably granzyme B mRNA. This is accomplished by hybridizing the nucleic acid, preferably at stringent conditions, in a sample with oligonucleotide probes that is specific for the granzyme B mRNA. Nucleic acid samples used in the methods and assays of the present invention may be prepared by any available method or process. Methods of isolating total RNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part 1—Theory and Nucleic Acid Preparation, Tijssen, (1993) (editor) Elsevier Press. Such samples include RNA samples, but also include cDNA synthesized from a mRNA sample isolated from a cell or tissue of interest. Such samples also include DNA amplified from the cDNA, and an RNA transcribed from the amplified DNA. One of skill in the art would appreciate that it is desirable to inhibit or destroy RNase present in homogenates before homogenates can be used.

Nucleic acid hybridization simply involves contacting a probe and target nucleic acid under conditions where the probe and its complementary target can form stable hybrid duplexes through complementary base pairing (see U.S. Pat. No. 6,333,155 to Lockhart et al, which is incorporated herein by reference). Methods of nucleic acid hybridization are well known in the art. In a preferred embodiment, the probes are immobilized on solid supports such as beads, microarrays, or gene chips.

The hybridized nucleic acids are typically detected by detecting one or more labels attached to the sample nucleic acids and or the probes. The labels may be incorporated by any of a number of means well known to those of skill in the art (see U.S. Pat. No. 6,333,155 to Lockhart et al, which is incorporated herein by reference). Commonly employed labels include, but are not limited to, biotin, fluorescent molecules, radioactive molecules, chromogenic substrates, chemiluminescent labels, enzymes, and the like. The methods for biotinylating nucleic acids are well known in the art, as are methods for introducing fluorescent molecules and radioactive molecules into oligonucleotides and nucleotides.

Although antibodies and nucleic acid probes are specifically disclosed herein, any molecule that specifically binds granzyme B protein or mRNA can be used to detect granzyme B upregulation in manners similar to those of the antibodies or nucleic acid probes. Specific binding reactions are taught, e.g. in WO 2008/021055; and U.S. Pat. Nos. 7,321,829; 7,267,992; 7,214,346; 7,138,232; 7,153,681; 7,026,002; 6,891,057; 6,589,798; 5,939,021; 5,723,345; and 5,710,006; which are incorporated herein by reference.

Detection methods for specific binding reactions, particularly for immunoassays and the nucleic acid assays, are well known for fluorescent, radioactive, chemiluminescent, chromogenic labels, as well as other commonly used labels. Briefly, fluorescent labels can be identified and quantified most directly by their absorption and fluorescence emission wavelengths and intensity. A microscope/camera setup using a light source of the appropriate wavelength is a convenient means for detecting fluorescent labels. Radioactive labels may be visualized by standard autoradiography, phosphor image analysis or CCD detector. Other detection systems are available and known in the art.

In another embodiment, because granzyme B is an enzyme, its detection can be effected through substrate degradation. In this embodiment, a sample is brought in contact with a substrate for granzyme B. The degradation of the substrate is measured which indirectly yields the levels for granzyme B. In this case, the higher the degradation rate the higher the levels of granzyme B present. Substrates for granzyme B are commercially available, e.g., through Oncolmmunin, Inc., Gaithersburg, Md.; CalBiochem, San Diego, Calif.; and A. G. Scientific, Inc., San Diego, Calif. Substrates for granzyme B and their methods are disclosed, e.g., in Koeplinger, et al., Protein Exp. Purif. 18:378, 2000; Karahashi et al., Biol. Pharm. Bull. 23:140, 2000; Harris, et al., J. Biol. Chem. 273:27364, 1998; Thornberry et al., J. Biol. Chem. 272:17907, 1997; Harris et al., J. Biol. Chem. 273:27364, 1998; and Thornberry et al., J. Biol. Chem. 272, 17907, 1997; which are incorporated herein by reference. The substrates or its enzymatic products can be detected fluorometrically or colormetrically.

### Use of Granzyme B in Platelets as Targets for Treating Sepsis

In an embodiment, the present invention provides methods for reducing cellular apoptosis by preventing or reducing or inhibiting platelet aggregation, and thus, reducing contact of GzmB-platelets with cells. The method involves treating cells with a compound effective to prevent, reduce, or inhibit platelet aggregation.

By reducing apoptosis, the method can also be used to treat sepsis. Compounds or drugs that are effective in preventing or reducing or inhibiting platelet aggregation can be administered to a septic animal to treat, alleviate, or ameliorate the symptom of sepsis. The compound or drug may be administered to an animal, preferably mammals such as humans, in need thereof as a pharmaceutical or veterinary composition, such as tablets, capsules, solutions, or emulsions. The compounds effective to prevent, reduce, or inhibit platelet aggregation include, but are not limited to, GP2a3b antagonists, ADP receptor/P2Y12 inhibitors, prostaglandin analogues (PGI2), COX inhibitors, thromboxane inhibitors, or phosphodiesterase inhibitors. Examples of GP2a3b antagonists are epifibitide, tirofiban, and abciximab. ADP receptor/P2Y12 inhibitors can be thienopyridines, such as clopidogrel, prasugrel, and ticlopidine. Examples of prostaglandin analogue include beraprost, prostacyclin, iloprost, and treprostinil. Examples of COX inhibitors are asprin, aloxiprin, carbasalate calcium, indobufen, and triflusal. Examples of thromboxane inhibitors include thromboxane synthase inhibitors such as dipyridamole and picotamide; and receptor antagonist such as terutroban. Other compounds effective to reduce apoptosis include anagrelide, heparin, cilostazol, and dipyridamole.

The terms “treating” or “alleviating” or “ameliorating” and similar terms used herein, include prophylaxis and full or partial treatment. The terms may also include reducing symptoms, ameliorating symptoms, reducing the severity of symptoms, reducing the incidence of the disease, or any other change in the condition of the patient, which improves the therapeutic outcome.

The administration of the drug can be through any known and acceptable route. Such routes include, but are not necessarily limited to, oral, via a mucosal membrane (e.g., nasally, via inhalation, rectally, intrauterally or intravaginally, sublingually), intravenously (e.g., intravenous bolus injection, intravenous infusion), intraperitoneally, and subcutaneously. Administering can likewise be by direct injection to a site (e.g., organ, tissue) containing a target cell (i.e., a cell to be treated). Furthermore, administering can follow any number of regimens. It thus can comprise a single dose or dosing of the drug, or multiple doses or dosings over a period of time. Accordingly, treatment can comprise repeating the administering step one or more times until a desired result is achieved. In embodiments, treating can continue for extended periods of time, such as weeks, months, or years. Those of skill in the art are fully capable of easily developing suitable dosing regimens for individuals based on known parameters in the art. The methods thus also contemplate controlling, but not necessarily eliminating, sepsis. The preferred routes of administration in accordance with the present invention are intravenous, intramuscular, subcutaneous, per os, per rectum, and intranasal.

The amount to be administered varies depending on the subject, stage of the disease, age of the subject, general health of the subject, and various other parameters known and routinely taken into consideration by those of skill in the medical arts. As a general matter, a sufficient amount of the drug will be administered in order to make a detectable change in the symptom of sepsis. Suitable amounts are disclosed herein, and additional suitable amounts can be identified by those of skill in the art without undue or excessive experimentation.

The drug is administered in a form that is acceptable, tolerable, and effective for the subject. Numerous pharmaceutical forms and formulations for biologically active agents are known in the art, and any and all of these are contemplated by the present invention. Thus, for example, the drug can be formulated in oral solution, a caplet, a capsule, an injectable, an infusible, a suppository, a lozenge, a tablet, a cream or salve, an inhalant, and the like.

Those of ordinary skill in the art will readily optimize effective dosages and administration regimens as determined by good medical practice and the clinical condition of the individual patient.

The frequency of dosing will depend on the pharmacokinetic parameters of the compounds and the routes of administration. The optimal pharmaceutical formulation will be determined by one of skill in the art depending on the route of administration and the desired dosage. Such formulations may influence the physical state, stability, rate of in vivo release and rate of in vivo clearance of the administered agents. Depending on the route of administration, a suitable dose is calculated according to body weight, body surface areas or organ size. The availability of animal models is particularly useful in facilitating a determination of appropriate dosages of a given therapeutic. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Typically, appropriate dosages are ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions. Those studies, however, are routine and within the level of skilled persons in the art.

It will be appreciated that the compositions and treatment methods of the invention are useful in fields of human medicine and veterinary medicine. Thus, the subject to be treated is a mammal, such as a human or other mammalian animal. For veterinary purposes, subjects include for example, farm animals including cows, sheep, pigs, horses and goats, companion animals such as dogs and cats, exotic and/or zoo animals, and laboratory animals including mice, rats, rabbits, guinea pigs and hamsters.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### Example

### Methods

Animals

Mice were purchased from Jackson Laboratories (Bar Harbor, Me., USA) and housed and bred in a conventional animal facility. All experiments were approved by our Institutional Animal Care and Use Committee. Cecal ligation and puncture was performed on male 8-12 week old mice at time=0 hours as previously described (20). Briefly, under isoflurane anesthesia with spontaneous ventilation, the cecum was exposed through a 1-cm-long midline abdominal incision, ligated loosely with 4-0 silk ties (Ethicon, Cornelia, Ga., USA), and punctured twice proximally with an 18-gauge needle. Fecal material was expressed and the bowel replaced in the abdomen. The incision was closed with 4-0 nylon sutures. Mice were resuscitated with 4 ml/100 g of body weight of subcutaneous saline.

Platelet Isolation

Intra-cardiac blood was drawn directly into sodium citrate (Becton-Dickinson, Franklin Lakes, N.J., USA) and immediately centrifuged at 770 rpm for 10 minutes at 25° C. Platelets were isolated from platelet-rich plasma by a single high-speed centrifugation over Ficoll-Paque™ Plus (GE Healthcare Bio-Sciences Corporation, Piscataway, N.J., USA). Microscopy of smears of platelet isolates showed >90% platelet purity. Platelets intended for mRNA studies were immediately placed in Trizol® (Invitrogen, Carlsbad, Calif., USA). Platelets intended for functional studies were filtered through a 10 mL sepharose 2B gel column to remove extraneous proteins as described by Vollmar, et al (21). Platelet concentrations were measured using a manual hemocytometer and concentrations equalized between samples by diluting with PBS.

Megakaryocyte Isolation

Murine megakaryocytes were isolated from mouse tibial and femoral bone marrow by flushing with Iscove's Modified Dulbecco's Medium (IMDM). The resulting marrow suspension was treated and passed through StemSep® magnetic gravity columns (StemCell Technologies, Vancouver, BC, Canada) according to the manufacturer's protocol using biotin-labeled anti-CD42d antibodies for positive selection. Purity was confirmed by light microscopy with Wright's stain (Sigma-Aldrich, St. Louis, Mo., USA). mRNA was isolated as described for platelets.

Splenectomy

Healthy control spleens were removed and immediately ground through a 40 μm mesh cell strainer. Splenocytes were centrifuged, washed, and layered over Ficoll-Paque™ Plus (GE Healthcare Bio-Sciences). CD4⁺ cells were isolated using StemSep® magnetic gravity columns (StemCell) according to the manufacturer's protocol.

Expression Profiling

Expression values were calculated using the dChip difference model probe set algorithm (http://biosunl.harvard.edu/complab/dchip/) and Probe Logarithmic Intensity Error Estimation (PLIER) (Affymetrix, Santa Clara, Calif.) algorithm. dChip and PLIER signals were imported into Hierarchical Clustering Explorer (HCE) (22) and the resulting unsupervised clusters were examined visually for appropriate grouping of profiles. The signals from the algorithm with the most appropriate profile grouping were used for all subsequent analyses within each species (i.e. murine=dChip, human=PLIER) and imported into GeneSpring GX (Agilent Technologies, Santa Clara, Calif., USA). The murine dataset (NCBI GEO Record #GSE10343) and human dataset (NCBI GEO Record #GSE 10361) were normalized within each chip to the 50th percentile and per gene to control chips. Using the cross-gene error model without multiple testing corrections, one-way ANOVA (p<0.001) generated a list of differentially expressed probe sets over time.

qRT-PCR

cDNA was synthesized using the SuperScript™ III First-Strand Synthesis System (Invitrogen) per the manufacturer's protocol. DNA primers (Invitrogen) were designed according to known gene sequences as follows: granzyme A (Forward) 5′-GAA CCA CTG CTA CTC GGC ATC TGG [FAM]TC-3′ (SEQ ID NO: 1); granzyme A (Reverse) 5′-CAG AAA TGT GGC TAT CCT TCA CC-3′ (SEQ ID NO:2); granzyme B (Forward) 5′-GAC GAT CCT GCT CTG ATT ACC CAT CG[FAM] C-3′ (SEQ ID NO: 3); granzyme B (Reverse) 5′-TCA GAT CCT GCC ACC TGT CCT A-3′ (SEQ ID NO: 4). GAPDH-containing wells served as positive controls and polymerase-free wells as negative controls. Reactions were run using an ABI PRISM® 7900HT PCR instrument (Applied Biosystems, Foster City, Calif., USA) and relative gene expression levels were calculated using Sequence Detection System 2.2 Software (Applied Biosystems). Expression values were normalized relative to sample GAPDH mRNA expression.

Detection of Apoptosis

CD4⁺ splenocytes from healthy control mice were co-incubated with platelets isolated from control or septic mice for 90 minutes at 37° C. and 5% CO₂ with or without platelet pre-treatment with 10 ng/mL of recombinant TNFα (Sigma-Aldrich) for 90 minutes. Splenocyte apoptosis was evaluated by TiterTACS™ (Trevigen, Gaithersburg, Md., USA), a quantitative colorimetric assay for in situ detection of DNA fragmentation. All samples were run in triplicate according to the manufacturer's protocol with data normalized to negative and nuclease-induced positive controls.

Statistical Analysis

Data were maintained in Microsoft Excel 2007 (Redmond, Wash., USA). Statistical significance was tested with SPSS15 (SPSS, Chicago, Ill., USA) using paired or un-paired T-tests. Results are reported as mean±standard error of the mean (SEM) unless otherwise specified.

### Results

Sepsis Induces Platelet Cell Death Gene Expression

All mice that underwent cecal ligation and puncture (CLP) developed signs and symptoms consistent with peritoneal sepsis including decreased grooming, lethargy, and gross pathologic peritonitis at sacrifice. These mice developed significant weight loss over 48 hours (mean±SEM_{0 h versus 48 h}: −14.8±1.6%; p<0.0001). Fourteen out of the 96 mice studied (14.6%) expired between 6 and 48 hours status post CLP and were not included in the final analyses.

Expression profiles [Mouse 430 plus 2.0 GeneChips® (Affymetrix, Santa Clara, Calif., USA)] of platelet mRNA pooled from 5 mice at each time point (0-naïve, 24, and 48 hours status post CLP) showed 59 probe sets, representing 56 unique genes (shown in Table 1), that were differentially regulated over the time interval studied. These genes were primarily related to gene ontology biological process groups previously well-described in the response to sepsis: cell adhesion, cell growth regulation, chemotaxis, inflammatory and immune responses, proteolysis, and signal transduction. Of these, 6 probe sets belonged to the gene ontology molecular function group for cell death (GO:0008219). In particular, between 0 and 48 hours granzymes A and B, potent cytotoxic serine proteases, were >100-fold up-regulated (fold change=549.6 and 141.3 respectively).

TABLE 1
Differentially regulated probe sets (n = 59) between 0 hour controls and septic
mice at 24 and 48 hours status post CLP

| | 24 | 48 | | | |
|---|---|---|---|---|---|
| | Hour | Hour | | | |
| Affymetrix | Fold | Fold | Genbank | | |
| Probe Set ID | Change | Change | ID | Gene Symbol | Gene Name |
| 1427747_a_at | 1593.0 | 540.1 | X14607 | Lcn2 | lipocalin 2 |
| 1440865_at | 276.4 | 202.3 | BB193024 | Ifitm6 | interferon induced |
| | | | | | transmembrane protein 6 |
| 1419764_at | 189.6 | 181.6 | NM_009892 | Chi313 | chitinase 3-like 3 |
| 1442339_at | 185.7 | 606.5 | BB667930 | MGI: 3524944 | stefin A2 like 1 |
| 1417898_a_at | 156.7 | 549.6 | NM_010370 | Gzma | granzyme A |
| 1418809_at | 153.0 | 530.7 | NM_011087 | Piral | paired-Ig-like receptor A1 |
| 1449984_at | 137.5 | 206.3 | NM_009140 | Cxcl2 | chemokine (C-X-C |
| | | | | | motif) ligand 2 |
| 1451563_at | 128.4 | 1831.0 | AF396935 | Emr4 | EGF-like module |
| | | | | | containing, mucin-like, |
| | | | | | hormone receptor-like |
| | | | | | sequence 4 |
| 1456250_x_at | 126.3 | 324.6 | BB533460 | Tgfbi | transforming growth |
| | | | | | factor, beta induced |
| 1422013_at | 120.3 | 759.8 | NM_011999 | Clec4a2 | C-type lectin domain |
| | | | | | family 4, member a2 |
| 1436530_at | 109.6 | 287.0 | AA666504 | | CDNA clone |
| | | | | | MGC: 107680 |
| | | | | | IMAGE: 6766535 |
| 1450826_a_at | 104.9 | 524.0 | NM_011315 | Saa3 | serum amyloid A 3 |
| 1419394_s_at | 104.6 | 48.6 | NM_013650 | S100a8 | S100 calcium binding |
| | | | | | protein A8 (calgranulin A) |
| 1424254_at | 98.9 | 86.1 | BC027285 | Ifitm1 | interferon induced |
| | | | | | transmembrane protein 1 |
| 1442798_x_at | 93.7 | 104.1 | BB324660 | Hk3 | hexokinase 3 |
| 1456223_at | 93.2 | 246.8 | BF322016 | | Transcribed locus |
| 1416635_at | 83.0 | 683.2 | NM_020561 | Smpd13a | sphingomyelin |
| | | | | | phosphodiesterase, |
| | | | | | acid-like 3A |
| 1437478_s_at | 81.2 | 200.3 | AA409309 | Efhd2 | EF hand domain |
| | | | | | containing 2 |
| 1422953_at | 77.8 | 62.0 | NM_008039 | Fpr-rs2 | formyl peptide receptor, |
| | | | | | related sequence 2 |
| 1436202_at | 76.1 | 160.7 | AI853644 | Malat1 | metastasis associated |
| | | | | | lung adenocarcinoma |
| | | | | | transcript 1 (non-coding |
| | | | | | RNA) |
| 1419709_at | 71.8 | 314.9 | NM_025288 | Stfa3 | stefin A3 |
| 1450808_at | 68.2 | 125.5 | NM_013521 | Fpr1 | formyl peptide receptor 1 |
| 1430700_a_at | 67.7 | 309.5 | AK005158 | Pla2g7 | phospholipase A2, |
| | | | | | group VII (platelet- |
| | | | | | activating factor |
| | | | | | acetylhydrolase, |
| | | | | | plasma) |
| 1448756_at | 67.5 | 23.5 | NM_009114 | S100a9 | S100 calcium binding |
| | | | | | protein A9 (calgranulin B) |
| 1420331_at | 65.8 | 251.8 | NM_019948 | Clec4e | C-type lectin domain |
| | | | | | family 4, member e |
| 1420330_at | 64.9 | 220.2 | NM_019948 | Clec4e | C-type lectin domain |
| | | | | | family 4, member e |
| 1423346_at | 63.2 | 272.6 | AV286991 | Degs1 | degenerative |
| | | | | | spermatocyte homolog |
| | | | | | 1 (Drosophila) |
| 1418722_at | 62.4 | 28.6 | NM_008694 | Ngp | neutrophilic granule |
| | | | | | protein |
| 1429900_at | 62.0 | 286.4 | BM241296 | 5330406M23Rik | RIKEN cDNA |
| | | | | | 5330406M23 gene |
| 1434773_a_at | 57.7 | 137.9 | BM207588 | Slc2a1 | solute carrier family 2 |
| | | | | | (facilitated glucose |
| | | | | | transporter), member 1 |
| 1420671_x_at | 57.0 | 413.4 | NM_029499 | Ms4a4c | membrane-spanning 4- |
| | | | | | domains, subfamily A, |
| | | | | | member 4C |
| 1419598_at | 55.4 | 276.6 | NM_026835 | Ms4a6d | membrane-spanning 4- |
| | | | | | domains, subfamily A, |
| | | | | | member 6D |
| 1421392_a_at | 53.8 | 140.5 | NM_007464 | Birc3 | baculoviral IAP |
| | | | | | repeat-containing 3 |
| 1418189_s_at | 53.2 | 197.8 | AF146523 | Malat1 | metastasis associated |
| | | | | | lung adenocarcinoma |
| | | | | | transcript 1 (non-coding |
| | | | | | RNA) |
| 1435761_at | 51.2 | 322.8 | AW146083 | Stfa3 | stefin A3 |
| 1419599_s_at | 49.6 | 362.9 | NM_026835 | Ms4a11 | membrane-spanning 4- |
| | | | | | domains, subfamily A, |
| | | | | | member 11 |
| 1421408_at | 49.3 | 246.7 | NM_030691 | Igsf6 | immunoglobulin |
| | | | | | superfamily, member 6 |
| 1418204_s_at | 46.1 | 282.2 | NM_019467 | Aif 1 | allograft inflammatory |
| | | | | | factor 1 |
| 1420394_s_at | 40.3 | 89.0 | U05264 | Gp49a; Lilrb4 | glycoprotein 49 A; |
| | | | | | leukocyte |
| | | | | | immunoglobulin-like |
| | | | | | receptor, subfamily B, |
| | | | | | member 4 |
| 1416530_a_at | 39.0 | 168.2 | BC003788 | Pnp | purine-nucleoside |
| | | | | | phosphorylase |
| 1437584_at | 38.8 | 158.8 | BE685667 | | Transcribed locus |
| 1419647_a_at | 38.6 | 109.6 | NM_133662 | Ier3 | immediate early |
| | | | | | response 3 |
| 1419060_at | 35.2 | 141.3 | NM_013542 | Gzmb | granzyme B |
| 1448123_s_at | 33.9 | 129.3 | NM_009369 | Tgfbi | transforming growth |
| | | | | | factor, beta induced |
| 1429954_at | 28.7 | 245.8 | AK014135 | Clec4a3 | C-type lectin domain |
| | | | | | family 4, member a3 |
| 1448061_at | 27.9 | 204.0 | AA183642 | Msr1 | macrophage scavenger |
| | | | | | receptor 1 |
| 1438943_x_at | 27.7 | 136.2 | AV308148 | Rpn1 | ribophorin I |
| 1439057_x_at | 23.3 | 292.2 | BB143557 | Zdhhc6 | zinc finger, DHHC |
| | | | | | domain containing 6 |
| 1448620_at | 22.2 | 77.9 | NM_010188 | Fcgr3 | Fc receptor, IgG, low |
| | | | | | affinity III |
| 1455899_x_at | 21.4 | 88.3 | BB241535 | Socs3 | suppressor of cytokine |
| | | | | | signaling 3 |
| 1447277_s_at | 20.9 | 630.1 | BB785407 | Pcyox1 | prenylcysteine oxidase 1 |
| 1419209_at | 20.5 | 407.7 | NM_008176 | Cxcl1 | chemokine (C-X-C |
| | | | | | motif) ligand 1 |
| 1433699_at | 17.7 | 58.8 | BM241351 | Tnfaip3 | tumor necrosis factor, |
| | | | | | alpha-induced protein 3 |
| 1455908_a_at | 16.3 | 212.3 | AV102733 | Scpep1 | serine carboxypeptidase 1 |
| 1457666_s_at | 14.8 | 67.8 | AV229143 | Ifi202b | interferon activated |
| | | | | | gene 202B |
| 1427076_at | 12.9 | 91.1 | L20315 | Mpeg1 | macrophage expressed |
| | | | | | gene 1 |
| 1420249_s_at | 8.8 | 94.7 | AV084904 | Ccl6 | chemokine (C-C motif) |
| | | | | | ligand 6 |
| 1416382_at | 6.1 | 101.0 | NM_009982 | Ctsc | cathepsin C |
| 1449193_at | 2.5 | 66.9 | NM_009690 | Cd5l | CD5 antigen-like |

Cell Death (GO: 0008219) genes (n = 6) noted in BOLD

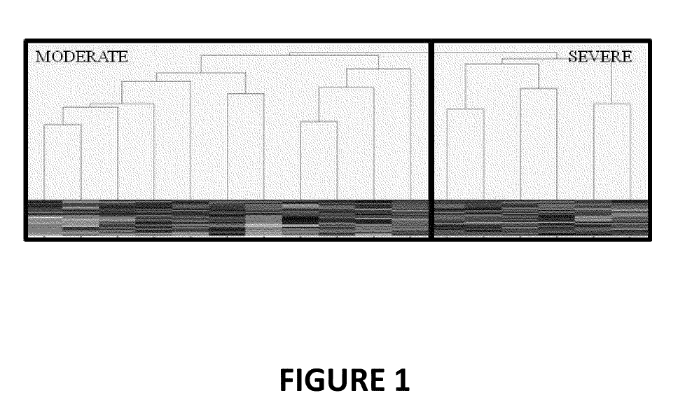

We explored expression of these cell death genes in human sepsis in an Institutional Review Board-approved study of septic children (n=17) between the ages of 1 and 18 (8.8±1.3) years. Nine participants (53%) were male. The diagnosis of sepsis was made using criteria adapted for pediatrics from the consensus definitions for sepsis (23-25). We collected clinical and laboratory data (i.e. the most extreme value in the prior 24 hours) over 72 hours. Relative clinical severity was determined by unsupervised clustering of all raw clinical and laboratory data in Hierarchical Clustering Explorer (HCE) (http://www.cs.umd.edu/hcil/hce/). (FIG. 1) The participants clearly clustered into two groups by clinical and laboratory variables. Group 1 (n=6) was designated “severe” because it had significantly higher severity of illness scores [i.e. mean Pediatric Risk of Mortality (PRISM) III (26) score (17.0±2.7 versus 4.5±1.1; p<0.001)] and longer hospital length of stay (45.5±10.6 versus 13.7±2.8 days; p=0.029). Group 2 (n=11) was designated “moderate” and was not significantly different from the severe group for other analyzed outcome variables including mortality and presence of shock.

As preliminary validation of the murine data, platelet mRNA from one exemplary severe and one exemplary moderate septic human subject was profiled using Human U133A GeneChips® (Affymetrix) and compared to platelet gene expression in three healthy young adult controls. There was no intent to conduct a statistically robust genome-wide assessment on this small group of samples but rather we focused on a cross-species screening for the six cell death genes identified in the murine study. Of those, only granzyme B was differentially-regulated over 72 hours (fold increase=2.9) in the severe subject. None of the other cell death genes studied showed differential expression in either group.

### Validation of Sepsis-Induced Changes in the Megakaryocyte-Platelet Transcriptional Axis

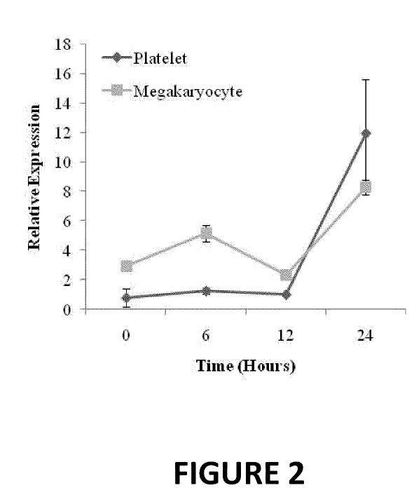

Quantitative reverse transcriptase polymerase chain reaction (qRT-PCR) was used to validate the murine platelet granzyme A and B up-regulation detected by microarray. We studied only the first 24 hours following induction of sepsis because the bulk of granzyme up-regulation seen by microarray occurred during this time period. In an independent cohort of septic mice (n=12; 3 mice per time point, non-pooled), granzyme B mRNA expression significantly increased from 0 to 24 hours (mean±SE_{0 h versus 24 h}: 0.77±0.61 versus 11.94±3.65; p=0.04). (FIG. 2) The expression of granzyme A mRNA was not significantly increased over that same time (mean±SE_{0 h versus 24 h}: 1.57±2.73 versus 2.61±4.53; p=0.11).

As platelets are anucleate and lack transcriptional machinery, we hypothesized that increased platelet granzyme B mRNA expression in sepsis could be further validated by simultaneous measurement in autologous megakaryocytes. Using qRT-PCR we measured platelet granzyme B mRNA expression in bone marrow megakaryocytes simultaneously acquired from the same mice used in the platelet qRT-PCR validation step. Megakaryocyte granzyme B mRNA relative expression increased significantly by 24 hours (mean±SE_{0 h versus 24 h}: 2.88±0.27 versus 8.25±0.52; p=0.05). Platelet granzyme B mRNA expression over time closely followed that of megakaryocytes. (FIG. 2) Megakaryocyte granzyme A mRNA expression did not change (mean±SEM_{0 h versus 24 h}: 3.18±0.54 versus 2.99±0.12; p=0.42).

Sepsis Induces Platelet Granzyme B Protein Expression

To determine if granzyme B mRNA up-regulation translates to increased granzyme B protein expression, additional citrated whole blood was collected from septic and control mice. It was fixed with 1% paraformaldehyde, permeabilized, and intracellularly stained with anti-granzyme B (clone 16G6; eBioscience, San Diego, Calif., USA) using appropriate isotype and negative (unlabeled) controls. Flow cytometry data were generated on a FACSCalibur™ System (BD Biosciences, San Jose, Calif., USA), gating on CD61⁺ (clone 2C9.G2; BD) platelets, and analyzed using FlowJo 7.2 (Tree Star, Inc., Ashland, Oreg., USA). Platelets from septic mice (n=9) showed an increase in intracellular granzyme B protein expression after 24 hours (mean±SEM_{0 h versus 24 h}: 4.4±1.3 versus 19.6±6.3%; p=0.039). Additional platelet activation with tumor necrosis factor (TNF) α did not alter intracellular granzyme B (data not shown).

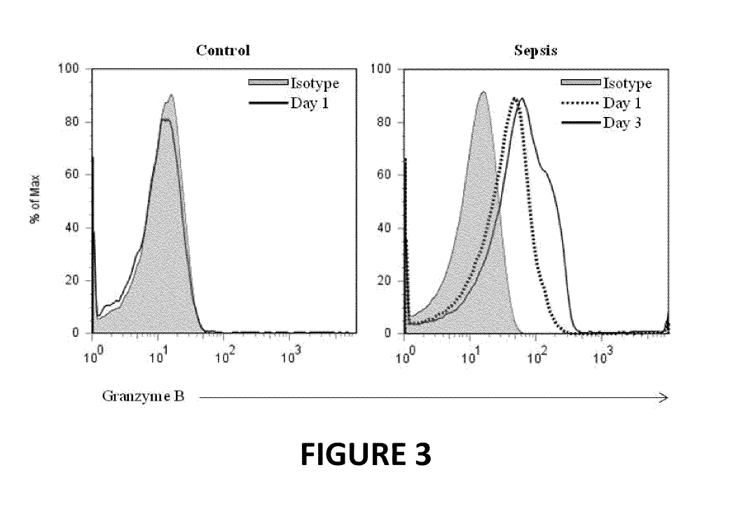

In a cross-species validation step, citrated whole blood from septic and healthy children was studied in a similar manner. In this case, flow cytometry data were generated on CD61⁺ (clone VI-PL2; BD) platelets stained for intracellular granzyme B (clone GB11; BD). Granzyme B was measured in one “severe” and three “moderate” subjects one and three days following admission for sepsis and compared to similarly-aged healthy control children (n=10) having blood drawn for routine testing. Platelets from the severe subject expressed intracellular granzyme B at both day one (49.7%) and day three (44.3%). (FIG. 3) Only one of the moderate septic subjects expressed any granzyme B and only at day three (24.0%). There was no measurable intracellular granzyme B in platelets from the control children. In addition, platelet activation state (i.e. CD62P⁺) did not affect granzyme B expression. Further, we did not detect surface expression of other apoptosis inducing proteins [i.e. Fas ligand (FasL), interleukin (IL) 1β, TNFα, and TNF-related apoptosis-inducing ligand (TRAIL)] on platelets from the septic children.

Platelets are Lymphotoxic Effectors in Sepsis Via Granzyme B

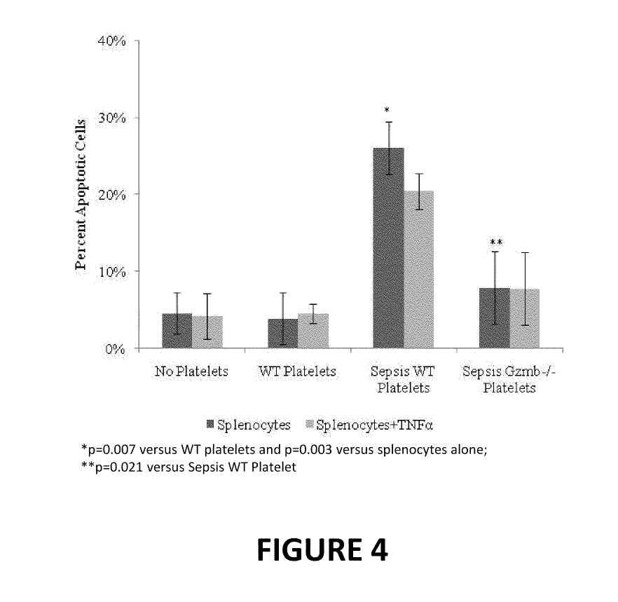

Our finding of granzyme B in platelets from septic mice and humans caused us to hypothesize that platelets could be lymphotoxic in this scenario. To study this question, platelets from mice 18 hours status post CLP were co-incubated with CD4⁺ splenocytes isolated from healthy control mice. Platelets from septic wild-type (i.e. C57BL6) mice induced marked splenocyte apoptosis compared to platelets from sham wild-type mice (rate of apoptosis=26.0±3.4 versus 3.9±3.4%; p=0.007). (FIG. 4) This co-incubation experiment was repeated with platelets from septic granzyme B null (−/−) mice (i.e. B6.129S2-Gzmb^{tmlLey}). In this case, there was a complete lack of induced splenocyte apoptosis by septic platelets. Notably, wild-type platelets further activated by TNFα had no more lymphotoxicity (4.5±1.3%; p=0.88) than non-activated control platelets. (FIG. 4)

### Discussion

Sepsis-related mortality results in part from immunodeficiency secondary to profound lymphoid apoptosis.(1, 2, 27-30) The biological mechanisms responsible for this extensive lymphocyte cell death is not understood but has been attributed in part to direct pathogen signaling through toll-like receptors and MyD88.(31) However, in these studies we explored the possibility that platelets play a direct role in this process by conducting time series studies in a murine experimental model of sepsis. Microarrays were used as an initial screening tool to hypothesize that responses of platelets to systemic perturbations in sepsis could lead to changes in mRNA expression of cell death-associated genes. This model was then tested through a series of mouse and human studies. Our experiments led us to characterize sepsis-induced changes in the megakaryocyte-platelet transcriptional axis and present a novel finding that the resulting platelets are strongly lymphotoxic. Second, using platelets from a murine induced-sepsis model we identified the serine protease, granzyme B, as the cause of this lymphotoxicity.

The granzymes are a group of cytotoxic serine proteases that are most commonly secreted within cytotoxic granules by natural killer (NK) and cytotoxic T lymphocytes.(32) Granzyme B is the most well-characterized of these proteases (the other human granzymes include A, H, K, and M) and has multiple known caspase targets and a growing list of caspase-independent substrates, including poly(ADP-ribose) polymerase (PARP)(33) and fibroblast growth factor receptor-1 (FGFR1).(34) Granzyme B typically enters target cells through a channel of co-released perforin (35) but can also enter independently.(36-38) Once in the target cell cytoplasm granzyme B cleaves several intracellular pro-apoptotic cysteine proteases, the most prominent and best-studied being caspase 3.(35) Alternatively, granzyme B has been shown to induce apoptosis via Bid-induced mitochondrial damage.(39-41) It is important to note that granzyme B has been shown to induce cell death by caspase- and non-caspase-mediated mechanisms simultaneously.(34, 42) In addition, Wong et al. showed that granzyme B is among the transcripts up-regulated in whole blood from pediatric septic shock nonsurvivors compared to survivors.(43)

Our experiments showed that platelets are in fact strongly lymphotoxic due to granzyme B in sepsis. Our results build upon previous research demonstrating significant inter-regulatory interactions between platelets and lymphocytes in a variety of inflammatory disease states, particularly with respect to adaptive immunity. For instance, platelet CD40 has been shown to bind to T lymphocyte CD40 ligand inducing platelet release of CCL5 which further activates T lymphocytes and thus, amplifies the immune response.(44) In particular in sepsis, platelet-derived microparticles have been shown to be cytotoxic against vascular endothelium (8-10) and smooth muscle.(10) However, to our knowledge, ours is the first study to examine acute changes in the platelet transcriptome in response to a disease insult. We found that megakaryocytes in the bone marrow respond to systemic sepsis and alter the transcriptome of platelets to include granzyme B.

The presence of granzyme B in platelets in sepsis raises intriguing questions, especially in light of the fact that platelet activation does not appear to impact its expression, implying there is no post-transcriptional regulation. First, it is possible that granzyme B serves a role in megakaryocyte caspase activation, which is critical for normal platelet formation.(45) If so, it is possible that in the hyper-thrombopoiesis of sepsis that megakaryocyte up-regulation of granzyme B mRNA results in inclusion of this transcript in platelets. An alternative is that platelet granzyme B represented an evolutionary advantage at some point. Granzyme B's ability to induce apoptosis through a wide variety of mechanisms makes it a likely mechanism to circumvent the immune evasion strategies of intracellular pathogens. In fact, there is evidence that granzyme B from cytotoxic T cells may play a role in defense against Toxoplasma gondii and Plasmodium species.(46, 47)

In summary, we conclude that platelets up-regulate granzyme B in murine and human sepsis. We further showed that platelets from septic mice induced marked apoptosis of healthy splenocytes ex vivo via granzyme B action. Our findings establish a conceptual advance in sepsis: Septic megakaryocytes produce platelets with acutely altered mRNA profiles and these platelets mediate lymphotoxicity via granzyme B. Given the contribution of lymphoid apoptosis to sepsis-related mortality, modulation of platelet granzyme B becomes an important new target for investigation and therapy.

### Example

### Methods

Animals

Wild type (i.e. C57BL6), perforin null (i.e. C57BL/6-PfptmlSdz), and granzyme B null mice (i.e. B6.12952-GzmBtmlLey) (Jackson Laboratories, Bar Harbor, Me.) were housed and bred in a conventional animal facility. Our Institutional Animal Care and Use Committee approved all experiments.

Experimental Sepsis and Sample Collection

Polymicrobial peritonitis and experimental sepsis was induced via a moderate-severity cecal ligation and puncture (CLP) in 7-10 week old male mice as we and others have previously described (Freishtat et al., Am J Respir Crit Care Med 2009; 179; Wichterman et al., J Surg Res 1980; 29:189-2011; Rittirsch et al., Nat Protocols 2008; 4:31-36). For mortality studies, we used a severe CLP model for rapid time to death (Rittirsch et al.) to minimize animal discomfort. In these mortality experiments mice were scored post-surgically in 2-hour intervals, starting at 16 hours, using a 15-point validated murine sepsis severity measure. Mice were sacrificed when a score of 10 [associated with >90% imminent mortality (Zantl et al., Infect Immun 1998; 66:2300-2309; Bougaki et al., Shock 2009; 3:281-290)] was reached. For non-mortality experiments, mice were sacrificed 18 hours post-surgery.

At the time of sacrifice, intra-cardiac blood was drawn into sodium citrate (Becton-Dickinson, Franklin Lakes, N.J.) and centrifuged for platelet-rich plasma at 770 rpm for 20 minutes at 25° C. Platelets were isolated by centrifugation and filtered through a 10 mL sepharose 2B gel column (Vollmar et al., Microcirculation 2003; 10:143-152). Platelet concentrations were measured and standardized using a manual hemocytometer.

Ex vivo Platelet-Splenocyte Co-Incubation

Non-septic wild type spleens were firmly pressed between two glass slides to express splenocytes, which were isolated by centrifugation through Ficoll-Paque™ Plus (GE Healthcare Bio-Sciences Corporation, Piscataway, N.J.). Splenocyte concentrations were measured and standardized using a manual hemocytometer and co-incubated ex vivo with platelets (from septic or healthy control mice) for 90 minutes at 37° C. and 5% CO2 in complete Dulbecco's Modified Eagle Medium (DMEM) (Invitrogen/GIBCO, Carlsbad, Calif.). For some experiments, a 0.4 μm semi-permeable membrane (Corning Inc., Corning, N.Y.) was used to physically separate platelets from splenocytes. In other experiments, platelet-splenocyte contact was pharmacologically inhibited using anti-aggregatory pretreatment with GPIIb/IIIa inhibitor, eptifibatide (4 μg/mL; Bachem, Torrance, Calif.) or anti-CD62p antibody (3 μg/mL; clone RB40.34; BD Biosciences, San Jose, Calif.) for 15 minutes.

Detection of Apoptosis

Splenocyte apoptosis in each experimental condition was quantified in cell suspensions by flow cytometry on a FACSCalibur™ (Becton, Dickinson and Company, San Jose, Calif.) and in tissue sections by immunohistochemistry on a Nikon Eclipse E 800 Microscope (Nikon Instruments Inc., Melville, N.Y.) with a Spot RT Slider Camera (Diagnostic Instruments Inc., Sterling Heights, Mich.).

Splenocyte suspension apoptosis was identified using FlowTACS™ (Trevigen, Gaithersburg, Md.), a TUNEL-based assay for detection of DNA fragmentation. Positive controls were generated with staurosporine (Sigma Life Sciences, St Louis, Mo.). CD4+ fractions were identified by fluorophore-labeled antibody staining (clone L3T4; eBiosciences, San Diego, Calif.). We used a Sulforhodamine FLICA-Apoptosis Detection Kit Pan-Caspase Assay (Immunochemistry Technologies, Bloomington, Minn.) to measure activated caspases in apoptotic cells. Immunohistochemistry was performed on frozen heart, lung, kidney, spleen, and liver sections (4-7 μm) stained with the TUNEL-based TACK® 2 TdT In Situ Apoptosis Detection Kit (Trevigen, Gaithersburg, Md., USA) according to the manufacturer's instructions.

Statistical Analyses

Flow cytometry data were analyzed using FlowJo 7.5 (Tree Star, Inc., Ashland, Oreg.). Data were maintained in Microsoft Excel 2010 (Microsoft, Redmond, Wash., USA). Statistical significance was tested using PASW 18 (SPSS, Chicago, Ill., USA).

### Results

Sepsis-Related Mortality is Reduced in the Absence of Granzyme B

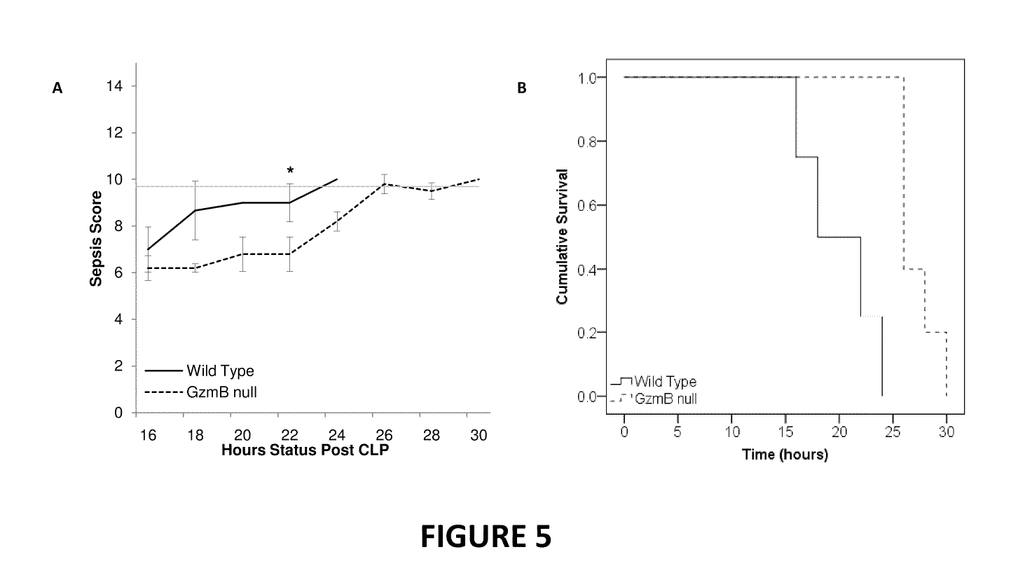

Following CLP-induced polymicrobial sepsis (severe model), granzyme B null mice (n=5) had lower sepsis scores than wild type mice (n=4) at every time point. (FIG. 5A) For example, at 22 hours, the mean±SEM wild type score was 9±0.8 while the granzyme B null score was 6.8±0.7 (P=0.04). At 24 hours post-CLP, the mortality rate of the granzyme B null mice was 0% while the mortality rate of the wild type mice was 100%. Kaplan-Meier survival analysis showed that granzyme B null mice survived longer following CLP than wild type mice (P=0.0019 by Cox Proportional Hazard Regression). (FIG. 5B)

Sepsis-Induced Spleen and Lung Apoptosis is Granzyme B-Dependent

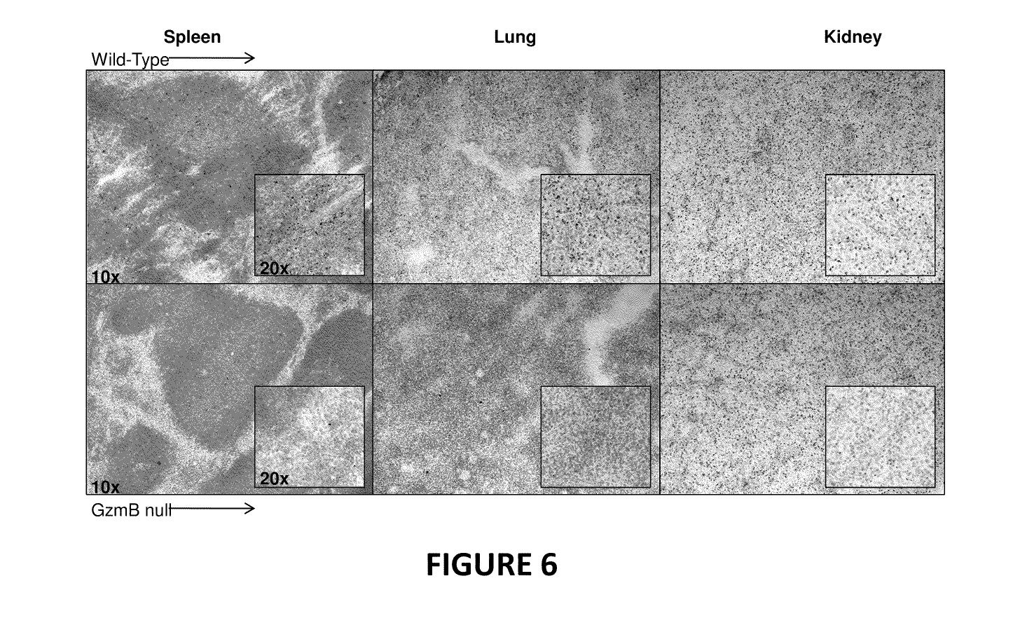

Spleen, lung, and kidney sections from wild type mice at 18 hours following CLP-induced polymicrobial sepsis (moderate model) were markedly TUNEL positive. (FIG. 6) In contrast, spleens and lungs from septic granzyme B null mice lacked TUNEL staining. Kidneys stained positive for TUNEL in both wild type and granzyme B null animals while heart and liver did not stain in either strain. Adjacent sections stained for platelet antigen CD41 (Rat anti-mouse CD41 antibody, BD Pharmingen, San Diego, Calif., USA) revealed similar abundant platelet accumulation in both lungs and spleens of septic wild type and granzyme B null mice.

Septic Platelets Induce Apoptosis in a Caspase-Mediated, Perforin-Independent Manner

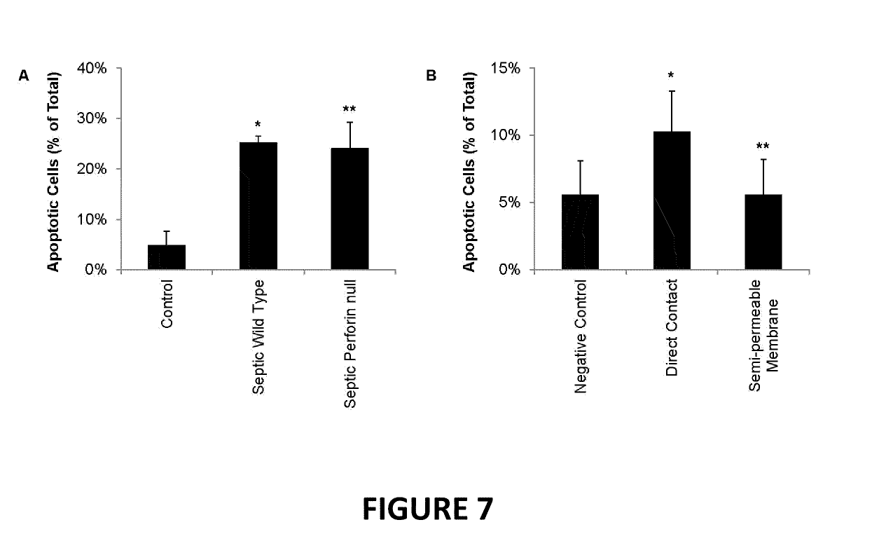

Granzyme B is known to target caspases in mice and humans (Trapani et al., J Biol Chem 1998; 273:27934-27938) and Bid-induced mitochondrial cell death pathways in humans only (Waterhouse et al., J Biol Chem 2005; 280:4476-4482; Waterhouse et al., Cell Death Differ 2006; 13:607-618; Waterhouse et al., Immunol Cell Biol 2006; 84:72-78). To confirm septic platelets induce apoptosis via a mechanism consistent with granzyme B action in mice, we used platelet:splenocyte co-incubations as an ex vivo model for this interaction. At 18 hours post-CLP, platelets from septic wild type mice induced more splenocyte apoptosis ex vivo than platelets from healthy wild type mice (25.1±1.4% versus 4.8±2.9%; p=0.0004) (FIG. 7A). The apoptotic splenocytes were almost entirely caspase+(i.e. >98%).

When formed in cytotoxic lymphocytes and natural killer cells, granzyme B typically enters target cells through a channel of co-released perforin (Trapani et al.) but can also enter independently (Choy et al., Arterioscler Thromb Vasc Biol 2004; 24:2245-2250). Therefore, we repeated the co-incubation experiments above with platelets from septic perforin null mice. In this condition, there was no change in percent splenocyte apoptosis by septic perforin null platelets (24.0±5.2%) compared to septic wild type platelets. (FIG. 7A).

Platelets Require Direct Physical Contact with Splenocytes to Induce Apoptosis

To determine if platelets can induce end-organ apoptosis in the absence of direct contact with the target cells (implying a microparticle-mediated process), septic platelets and healthy splenocytes were incubated as before, in suspension or separated by a semi-permeable membrane. Incubation across the membrane completely abrogated splenocyte apoptosis, as measured by both TUNEL (p<0.05) (FIG. 7B) and caspase to a rate indistinguishable from healthy controls. As before, apoptotic splenocytes were almost entirely caspase positive (i.e. >98%).

Platelet-Induced Splenocyte Apoptosis is Blocked by GPIIb/IIIa Inhibition

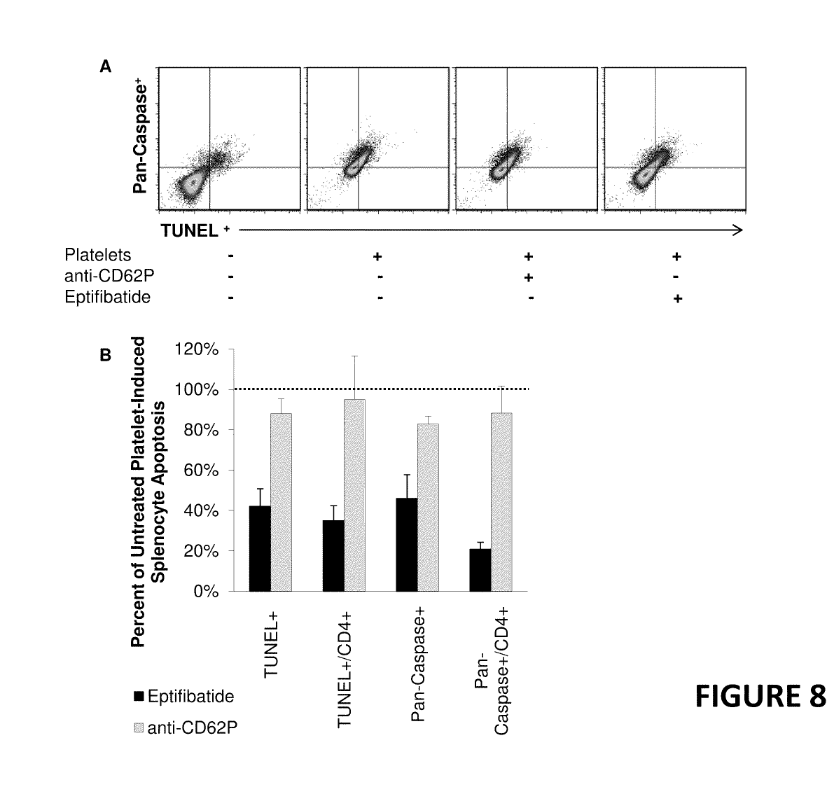

The finding that physical separation of septic platelets from splenocytes eliminated apoptosis raised the question whether pharmacologic separation would have the same effect. To that end, platelet aggregation was inhibited ex vivo with either a poor (anti-CD62P neutralizing antibody) or strong (eptifibatide) platelet aggregation inhibitor. Co-incubation of eptifibatide-exposed septic wild type platelets with healthy splenocytes significantly decreased splenocyte apoptosis overall and in the CD4+ fraction as compared to co-incubation with non-exposed septic platelets (overall=66.5±10.6% reduction, p=0.008; CD4+=85±20.7% reduction, p=0.026). (FIG. 8) No difference in apoptosis was observed for septic platelets pretreated with the anti-CD62P antibody. Although only eptifibitide is used in the present experiment, other antiplatelet drugs are expected to similarly reduce apoptosis.

### Discussion

Using an experimental model of murine sepsis we defined the site(s) of and mechanism(s) by which platelets induce end-organ apoptosis in sepsis. Platelet induced-apoptosis occurs in spleen and at least one non-lymphoid organ, lung. This granzyme B-mediated cytotoxicity requires direct contact between platelets and end-organ cells but is perforin-independent. Further, we exploited the therapeutic potential of the contact-dependent nature of platelet-induced splenocyte apoptosis by markedly reducing ex vivo apoptosis with eptifibatide, a GPIIb/IIIa receptor inhibitor of platelet aggregation. These findings extend our previous work identifying platelet granzyme B-based cytotoxicity in septic humans and mice (Freishtat et al., Am J Respir Crit Care Med 2009; 179).

Platelets are known to accumulate in both immune (splenic) (Sigurdsson et al., Critical Care Medicine 1992; 20:458-467) and non-immune organs (liver, lung, intestine) during sepsis (Sigurdsson et al.; Drake et al., Am J Pathol 1993; 142:1458-1470; Shibazaki et al., Infect Immun 1996; 64:5290-5294; Shizabaki et al., Infect Immun 1999; 67:5186-5191). Meanwhile, sepsis leads to apoptosis of both immune (lymphocytes) and non-immune cells (epithelial, endothelial, lung and intestine) (Coopersmith et al., JAMA 2002; 287:1716-1721; Hotchkiss et al., Crit Care Med 1997; 25:1298-1307; Mutunga et al., Am J Respir Crit Care Med 2001; 163:195-200). Lymphocyte apoptosis in sepsis is widespread, occurring in thymus, spleen and gut-associated lymphoid tissues and has been shown to be associated with worse outcome (LeTulzo et al., Shock 2002; 18:487-494; Inoue et al., Journal of Immunology 2010; 184:1401-1409; Chung et al., Shock 2010; 34:150-161). Increased levels of splenocyte apoptosis in particular reduce survival in animals after CLP (Hiramatsu et al., Shock 1997; 7) demonstrating the importance of our finding that absence of granzyme B leads to diminished splenocyte apoptosis. Herein we showed that sites of platelet aggregation (i.e. lung and spleen) also show increased levels of apoptosis in granzyme B containing, but not granzyme B null mice. Prior to this, the coincident accumulation of platelets in failing organs in sepsis (Drake et al., Am J Pathol 1993; 142:1458-1470; Shibazaki et al., Infect Immun 1996; 64:5290-5294; Shizabaki et al., Infect Immun 1999; 67:5186-5191; Schneider et al., Am Rev Respir Dis 1980; 122:445-451) raised cause-and-effect questions. Our findings suggest platelets are causative in this relationship.

In addition to determining sites of platelet granzyme B-induced apoptosis in sepsis, we also determined vital mechanistic aspects of this process. In its typical role, in cytotoxic lymphocytes, granzyme B is stored and then released from secretory granules (also frequently containing perforin) upon synapse formation with virus infected or transformed target cells, leading to induction of apoptotic cell death pathways (Hoves et al., J Leukoc Biol 2010; 87:237-243). Whether platelet granzyme B-mediated apoptosis proceeds in a similar fashion was unknown. We showed that platelet granzyme B-mediated apoptosis is perforin-independent and required direct contact between platelets and target cells. The requirement for direct contact between platelets and lymphocytes suggests that platelet-derived microparticles (which alone can be cytotoxic) (Azevedo et al., Endocr Metab Immune Disord Drug Targets 2006; 6:159-164; Gambim et al., Crit Care Med 2007; 11:R107; Janiszewski et al., Crit Care med 2004; 32:818-825) are not the primary initiator of this apoptosis.

The contact dependent nature of platelet-induced splenocyte apoptosis led us to hypothesize that inhibitors of platelet aggregation could potentially decrease target cell apoptosis during sepsis. In fact, we demonstrated that septic platelet treatment with the anti-platelet compound, eptifibatide, reduces splenocyte apoptosis ex vivo. Eptifibatide functions by acting as an antagonist to the plasma membrane glycoprotein GPIIb/IIIa, which is found solely on platelets and platelet progenitor cells. GPIIb/IIIa belongs to a large class of cell surface receptors known as integrins, which take part in cell adhesion (Phillips et al., Blood 1988; 71:831-843; Kieffer et al., Annu Rev Cell Biol 1990; 6:329-357; Phillips et al., Cell 1991; 65:359-362; Hynes, Cell 1992; 69:11-25). When platelets become activated, fibrinogen binds to multiple GPIIb/IIIa receptors, thereby bridging platelets and facilitating platelet aggregation. Eptifibatide, in particular, is an extremely effective inhibitor of platelet aggregation and is unique in the fact that it binds specifically to GPIIb/IIIa, with low affinity for other integrins (Phillips D, Scarborough R. Clinical pharmacology of eptifibatide. Am J Cardiol 1997; 80:11 B-20B).

With regards to sepsis, other GPIIb/IIIa antagonist compounds have been studied in animal models and in certain cases have been shown to decrease coagulation activation and subsequent endothelial dysfunction and tissue injury during septic shock (Pu et al., Crit Care Med 2001; 29:1181-1188; Lipcsey et al., Platelets 2005; 16:408-414; Taylor et al., Blood 1997; 89:4078-4084; Seidel et al., J Thromb Haemost 2009; 7:1030-1032). Our finding that pretreatment and subsequent co-incubation in the presence of eptifibatide decreases splenocyte apoptosis has potentially important implications and warrants further study in vivo for a protective role for anti-platelet compounds during sepsis. Such an experiment has been reported, although using retrospective data. Two studies, both from the same center, showed decreased mortality and decreased levels of MODS in examined of adults admitted to the ICU who were already receiving anti-platelet compounds (either aspirin, clopidogrel or a combination of the two) for other reasons (Winning et al., Crit Care Med 2010; 38:32-37; Winning et al., Platelets 2009; 20:50-57). Notable also was the fact that anti-platelet medications did not increase rates of bleeding.

Anti-aggregation of platelets and splenocytes (i.e. reduced platelet-splenocyte contact) is only one possible mechanism by which eptifibatide may act in this scenario. Another possible mechanism is outside-in signaling, a mechanism by which extracellular binding to integrins activates intracellular signaling pathways (Giancotti et al., Science 1999; 285:1028-1032), resulting in cytoskeletal rearrangements, and decreased platelet alpha granule release (Shattil et al., Blood 2004; 104:1606-1615). It is possible that eptifibatide inhibits the activation of intracellular signaling pathways, leading to a decrease in the release of alpha granules, which may contain apoptosis promoting proteins. This pathway's ability to participate in sepsis and MODS warrants further investigation.

In summary, during sepsis, platelet granzyme B-mediated apoptosis occurs in spleen and lung tissue. This process proceeds in a perforin-independent, caspase-mediated, and contact-dependent manner, which can be inhibited by the GPIIb/IIIa inhibitor eptifibatide. Inhibition of platelet aggregation via GPIIb/IIIa blockade may act to decrease both lymphocyte apoptosis and reduce MODS during sepsis. In our preclinical experiments, the absence of granzyme B resulted in less severe sepsis and extended survival. This builds on prior work demonstrating that granzyme B is upregulated in septic shock non-survivors (Wong et al., Physiol Genomics 2007; 30:146-155) and further solidifies the important role played by this enzyme in platelets during sepsis.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

### REFERENCES

1. Hotchkiss R S, Karl I E. The pathophysiology and treatment of sepsis. N Engl J Med 2003; 348:138-150.
2. Russell J A. Management of sepsis. N Engl J Med 2006; 355:1699-1713.
3. Angus D C, Linde-Zwirble W T, Lidicker J, Clermont G, Carcillo J, Pinsky M R. Epidemiology of severe sepsis in the united states: Analysis of incidence, outcome, and associated costs of care. Crit Care Med 2001; 29:1303-1310.
4. Kochanek K D, Smith B L. Deaths: Preliminary data for 2002. Natl Vital Stat Rep 2004; 52:1-47.
5. Hotchkiss R S, Nicholson D W. Apoptosis and caspases regulate death and inflammation in sepsis. Nat Rev Immunol 2006; 6:813-822.
6. Shibazaki M, Nakamura M, Endo Y. Biphasic, organ-specific, and strain-specific accumulation of platelets induced in mice by a lipopolysaccharide from escherichia coli and its possible involvement in shock. Infect Immun 1996; 64:5290-5294.
7. Drake T A, Cheng J, Chang A, Taylor F B, Jr. Expression of tissue factor, thrombomodulin, and e-selectin in baboons with lethal escherichia coli sepsis [published erratum appears in am j pathol 1993 aug; 143(2):649]. Am J Pathol 1993; 142:1458-1470.
8. Azevedo L C, Janiszewski M, Soriano F G, Laurindo F R. Redox mechanisms of vascular cell dysfunction in sepsis. Endocr Metab Immune Disord Drug Targets 2006; 6:159-164.
9. Gambim M H, Carmo A O, Marti L, Verissimo-Filho S, Lopes L R, Janiszewski M. Platelet-derived exosomes induce endothelial cell apoptosis through peroxynitrite generation: Experimental evidence for a novel mechanism of septic vascular dysfunction. Crit Care 2007; 11:R107.
10. Janiszewski M, Do Carmo A O, Pedro M A, Silva E, Knobel E, Laurindo F R. Platelet-derived exosomes of septic individuals possess proapoptotic nad(p)h oxidase activity: A novel vascular redox pathway. Crit Care Med 2004; 32:818-825.
11. Raghavachari N, Xu X, Harris A, Villagra J, Logun C, Barb J, Solomon M A, Suffredini A F, Danner R L, Kato G, et al. Amplified expression profiling of platelet transcriptome reveals changes in arginine metabolic pathways in patients with sickle cell disease. Circulation 2007; 115:1551-1562.
12. Dittrich M, Birschmann I, Pfrang J, Herterich S, Smolenski A, Walter U, Dandekar T. Analysis of sage data in human platelets: Features of the transcriptome in an anucleate cell. Thromb Haemost 2006; 95:643-651.
13. Hillmann A G, Harmon S, Park S D, O'Brien J, Shields D C, Kenny D. Comparative rna expression analyses from small-scale, single-donor platelet samples. J Thromb Haemost 2006; 4:349-356.
14. Ouwehand W H. Platelet genomics and the risk of atherothrombosis. J Thromb Haemost 2007; 5 Suppl 1:188-195.
15. Weyrich A S, Denis M M, Schwertz H, Tolley N D, Foulks J, Spencer E, Kraiss L W, Albertine K H, McIntyre T M, Zimmerman G A. Mtor-dependent synthesis of bcl-3 controls the retraction of fibrin clots by activated human platelets. Blood 2007; 109:1975-1983.
16. Weyrich A S, Dixon D A, Pabla R, Elstad M R, McIntyre T M, Prescott S M, Zimmerman G A. Signal-dependent translation of a regulatory protein, bcl-3, in activated human platelets. Proceedings of the National Academy of Sciences 1998; 95:5556-5561.
17. Zimmerman G A, Weyrich A S. Signal-dependent protein synthesis by activated platelets: New pathways to altered phenotype and function. Arterioscler Thromb Vasc Biol 2008; 28:s17-24.
18. Freishtat R J, Natale J, Cohen J S, Mojgani B, Benton A S, Ngor W M, Bradbury M, Bunaj C, Hoffman E P. Platelets as cytotoxic mediators in sepsis: Platelet granzyme b induces lymphocyte apoptosis. Journal of Investigative Medicine 2008; 56:807-808.
19. Freishtat R J, Natale J, Cohen J S, Sachdeva R, De Biase L M, Ngor E, Degnan A, Benton A S, Chow M, Kristosturyan E, et al. Platelet granzyme b expression is increased in sepsis: A potential mechanism for sepsis-associated endothelial apoptosis. Faseb Journal 2007; 21:A1125-A1125.
20. Wichterman K A, Baue A E, Chaudry I H. Sepsis and septic shock—a review of laboratory models and a proposal. J Surg Res 1980; 29:189-201.
21. Vollmar B, Slotta J E, Nickels R M, Wenzel E, Menger M D. Comparative analysis of platelet isolation techniques for the in vivo study of the microcirculation. Microcirculation 2003; 10:143-152.
22. Seo J, Bakay M, Chen Y W, Hilmer S, Shneiderman B, Hoffman E P. Interactively optimizing signal-to-noise ratios in expression profiling: Project-specific algorithm selection and detection p-value weighting in affymetrix microarrays. Bioinformatics 2004; 20:2534-2544.
23. Bone R C, Sibbald W J, Sprung C L. The accp-sccm consensus conference on sepsis and organ failure. Chest 1992; 101:1481-1483.
24. Proulx F, Fayon M, Farrell C A, Lacroix J, Gauthier M. Epidemiology of sepsis and multiple organ dysfunction syndrome in children. Chest 1996; 109:1033-1037.
25. Proulx F, Gauthier M, Nadeau D, Lacroix J, Farrell C A. Timing and predictors of death in pediatric patients with multiple organ system failure. Crit Care Med 1994; 22:1025-1031.
26. Pollack M M, Patel K M, Ruttimann U E. Prism iii: An updated pediatric risk of mortality score. Crit Care Med 1996; 24:743-752.
27. Hotchkiss R S, Tinsley K W, Karl I E. Role of apoptotic cell death in sepsis. Scand J Infect Dis 2003; 35:585-592.
28. Groesdonk H V, Wagner F, Hoffarth B, Georgieff M, Senftleben U. Enhancement of nf-{kappa}b activation in lymphocytes prevents t cell apoptosis and improves survival in murine sepsis. J Immunol 2007; 179:8083-8089.
29. Hotchkiss R S, Osmon S B, Chang K C, Wagner T H, Coopersmith C M, Karl I E. Accelerated lymphocyte death in sepsis occurs by both the death receptor and mitochondrial pathways. J Immunol 2005; 174:5110-5118.
30. Wesche D E, Lomas-Neira J L, Perl M, Chung C S, Ayala A. Leukocyte apoptosis and its significance in sepsis and shock. J Leukoc Biol 2005; 78:325-337.
31. Peck-Palmer O M, Unsinger J, Chang K C, Davis C G, McDunn J E, Hotchkiss R S. Deletion of myd88 markedly attenuates sepsis-induced t and b lymphocyte apoptosis but worsens survival. J Leukoc Biol 2008:jlb. 0807528.
32. Masson D, Tschopp J. A family of serine esterases in lytic granules of cytolytic t lymphocytes. Cell 1987; 49:679-685.

33. Froelich C J, Hanna W L, Poirier G G, Duriez P J, D'Amours D, Salvesen G S, Alnemri E S, Earnshaw W C, Shah G M. Granzyme b/perforin-mediated apoptosis of jurkat cells results in cleavage of poly(adp-ribose) polymerase to the 89-kda apoptotic fragment and less abundant 64-kda fragment. Biochem Biophys Res Commun 1996; 227:658-665.
34. Loeb C R, Harris J L, Craik C S. Granzyme b proteolyzes receptors important to proliferation and survival, tipping the balance toward apoptosis. J Biol Chem 2006; 281:28326-28335.
35. Trapani J A, Jans D A, Jans P J, Smyth M J, Browne K A, Sutton V R. Efficient nuclear targeting of granzyme b and the nuclear consequences of apoptosis induced by granzyme b and perforin are caspase-dependent, but cell death is caspase-independent. J Biol Chem 1998; 273:27934-27938.
36. Choy J C, Hung V H Y, Hunter A L, Cheung P K, Motyka B, Goping I S, Sawchuk T, Bleackley R C, Podor T J, McManus B M, et al. Granzyme b induces smooth muscle cell apoptosis in the absence of perforin: Involvement of extracellular matrix degradation. Arterioscler Thromb Vasc Biol 2004; 24:2245-2250.
37. Florian C K, Martin K, Edward F, Klaus D, Rainer R, Hauke L, Dieter E J. Killing of target cells by redirected granzyme b in the absence of perforin. FEBS letters 2004; 562:87-92.
38. Gondek D C, Lu L-F, Quezada S A, Sakaguchi S, Noelle R J. Cutting edge: Contact-mediated suppression by cd4+cd25+ regulatory cells involves a granzyme b-dependent, perforin-independent mechanism. J Immunol 2005; 174:1783-1786.
39. Waterhouse N J, Sedelies K A, Browne K A, Wowk M E, Newbold A, Sutton V R, Clarke C J, Oliaro J, Lindemann R K, Bird P I, et al. A central role for bid in granzyme b-induced apoptosis. J Biol Chem 2005; 280:4476-4482.
40. Waterhouse N J, Sedelies K A, Sutton V R, Pinkoski M J, Thia K Y, Johnstone R, Bird P I, Green D R, Trapani J A. Functional dissociation of deltapsim and cytochrome c release defines the contribution of mitochondria upstream of caspase activation during granzyme b-induced apoptosis. Cell Death Differ 2006; 13:607-618.
41. Waterhouse N J, Sedelies K A, Trapani J A. Role of bid-induced mitochondrial outer membrane permeabilization in granzyme b-induced apoptosis. Immunol Cell Biol 2006; 84:72-78.
42. Bredemeyer A J, Carrigan P E, Fehniger T A, Smith D F, Ley T J. Hop cleavage and function in granzyme b-induced apoptosis. J Biol Chem 2006; 281:37130-37141.
43. Wong H R, Shanley T P, Sakthivel B, Cvijanovich N, Lin R, Allen G L, Thomas N J, Doctor A, Kalyanaraman M, Tofil N M, et al. Genome-level expression profiles in pediatric septic shock indicate a role for altered zinc homeostasis in poor outcome. Physiol Genomics 2007; 30:146-155.
44. Danese S, de la Motte C, Reyes B M, Sans M, Levine A D, Fiocchi C. Cutting edge: T cells trigger cd40-dependent platelet activation and granular rantes release: A novel pathway for immune response amplification. J Immunol 2004; 172:2011-2015.
45. Clarke M C H, Savill J, Jones D B, Noble B S, Brown S B. Compartmentalized megakaryocyte death generates functional platelets committed to caspase-independent death. J Cell Biol 2003; 160:577-587.
46. Hurd H, Carter V. The role of programmed cell death in plasmodium-mosquito interactions. Int J Parasitol 2004; 34:1459-1472.
47. Gavrilescu L C, Denkers E Y. Apoptosis and the balance of homeostatic and pathologic responses to protozoan infection. Infect Immun 2003; 71:6109-6115.
48. Warkentin T E, Aird W C, Rand J H. Platelet-endothelial interactions: Sepsis, hit, and antiphospholipid syndrome. Hematology 2003; 2003:497-519.

What is claimed is:

1. A method for preventing or reducing apoptosis comprising the step of contacting cells with a compound effective to prevent, reduce, or inhibit platelet aggregation.

2. The method of claim 1, wherein the compound is an antiplatelet drug.

3. The method of claim 2, wherein the antiplatelet drug is a GP2a3b antagonist, a ADP receptor/P2Y12 inhibitor, a prostaglandin analogue (PGI2), a COX inhibitor, a thromboxane inhibitor, or a phosphodiesterase inhibitor.

4. The method of claim 3, wherein the GP2a3b antagonist is epifibitide, tirofiban, or abciximab.

5. The method of claim 3, wherein the ADP receptor/P2Y12 inhibitor is clopidogrel, prasugrel, or ticlopidine.

6. The method of claim 3, wherein the prostaglandin analogue is beraprost, prostacyclin, iloprost, or treprostinil.

7. The method of claim 3, wherein the COX inhibitor is asprin, aloxiprin, carbasalate calcium, indobufen, or triflusal.

8. The method of claim 3, wherein the thromboxane inhibitor is dipyridamole, picotamide, or terutroban.

9. A method for treating sepsis in a individual comprising the step of administering to an individual having a compound effective to prevent, reduce, or inhibit platelet aggregation.

10. The method of claim 9, wherein the compound is an antiplatelet drug.

11. The method of claim 10, wherein the antiplatelet drug is a GP2a3b antagonist, a ADP receptor/P2Y12 inhibitor, a prostaglandin analogue (PGI2), a COX inhibitor, a thromboxane inhibitor, or a phosphodiesterase inhibitor.

12. The method of claim 11, wherein the GP2a3b antagonist is epifibitide, tirofiban, or abciximab.

13. The method of claim 11, wherein the ADP receptor/P2Y12 inhibitor is clopidogrel, prasugrel, or ticlopidine.

14. The method of claim 11, wherein the prostaglandin analogue is beraprost, prostacyclin, iloprost, or treprostinil.

15. The method of claim 11 wherein the COX inhibitor is asprin, aloxiprin, carbasalate calcium, indobufen, or triflusal.

16. The method of claim 11, wherein the thromboxane inhibitor is dipyridamole, picotamide, or terutroban.

17. A method for screening for a drug candidate for the treatment of sepsis comprising the steps of
a. treating a cell sample with an agent;
b. adding granzyme B containing platelets to the treated cell sample; and
b. determining whether the agent is effective in preventing apoptosis of the cells when compared to controlled cells not treated with the compound.

18. The method of claim 17, wherein the agent is selected from the group consisting of proteins, peptides, small molecules, vitamin derivatives, and carbohydrates.

19. The method of claim 17, further comprising the step determining whether the agent is effective in preventing platelet aggregation.
